(19) United States
(12) Patent Application Publication
(10) Pub. No.: US 2014/0088502 A1
(43) Pub. Date: Mar. 27, 2014

(54) Multi-Needle Injection Apparatus and System for Delivering Pharmacological Agents to Biological Tissue

(71) Applicants: Robert G. Matheny, Norcross, GA (US); Bret Herscher, Cupertino, CA (US); Michael Perry, Los Gatos, CA (US)

(72) Inventors: Robert G. Matheny, Norcross, GA (US); Bret Herscher, Cupertino, CA (US); Michael Perry, Los Gatos, CA (US)

(21) Appl. No.: 14/031,630

(22) Filed: Sep. 19, 2013

Related U.S. Application Data
(60) Provisional application No. 61/704,634, filed on Sep. 24, 2012.

(51) Int. Cl. A61M 5/19; A61K 38/30; A61K 35/28; A61K 38/39; A61K 39/395; A61K 38/48; A61K 38/17; A61K 31/727; A61K 35/32; A61K 35/12; A61K 35/34; A61K 35/36; A61K 35/407; A61K 35/44; A61K 35/50; A61K 35/54; A61K 38/18

(52) U.S. Cl. CPC A61M 5/19; A61K 38/1866; A61K 38/1858; A61K 38/30; A61K 38/1825; A61K 38/1833; A61K 38/1808; A61K 35/28; A61K 38/39; A61K 39/395; A61K 38/48; A61K 38/1741; A61K 31/727; A61K 35/32; A61K 35/33; A61K 35/34; A61K 35/35; A61K 35/36; A61K 35/407; A61K 35/44; A61K 35/50; A61K 35/545
USPC 604/121; 514/8.1; 514/8.2; 514/8.5; 514/9.1; 514/9.5; 514/9.6; 424/93.7; 514/17.2; 424/130.1; 424/94.63; 514/20.9; 514/56

(57) ABSTRACT

An injector system for delivery of a pharmacological agent to biological tissue having a plurality of needles arranged in an array, a plurality of reservoirs configured to receive a pharmacological composition therein, the reservoirs being in fluid communication with a respective one of the plurality of needles, a pneumatic pressure source, a pneumatic drive system that is configured to induce a first discharge of the pharmacological composition out of the reservoirs and needles in a plurality of delivery modes, agent delivery volume control means for controlling volume of the pharmacological composition discharged from the needles, and a control system having actuation control means for controlling said pneumatic drive system. The control system is programmed to control the delivery modes.

### CROSS-REFERENCES TO RELATED APPLICATIONS

This application claims the benefit of U.S. Application No. 61/704,634, filed on Sep. 24, 2012.

### FIELD OF THE INVENTION

The present invention relates to methods and systems for treating biological tissue. More particularly, the present invention relates to injection methods and systems for delivering pharmacological agents and formulations to damaged and/or diseased biological tissue; particularly, cardiovascular tissue.

### BACKGROUND OF THE INVENTION

Myocardial infarction is a common presentation of ischemic heart disease/coronary artery disease. The World Health Organization estimated in 2004 that 12.2% of worldwide deaths occurred as a result of ischemic heart disease. Ischemic heart disease was also deemed the leading cause of death in middle to high income countries and second only to respiratory infections in lower income countries. The Global Burden of Disease: World Health Organization 2004 Update, Geneva (2008). Worldwide more than 3 million people present with a ST elevation myocardial infarction (STEMI) and 4 million people present with a non-ST elevation myocardial infarction (NSTEMI) a year. White, et al., Acute Myocardial Infarction, Lancet 372 (9638), pp. 570-84 (August 2008).

Rates of death from ischemic heart disease have slowed or declined in most high income countries, although cardiovascular disease still accounted for 1 in 3 of all deaths in the USA in 2008. Roger, et al., Executive summary: Heart Disease and Stroke Statistics—2012 update: A report from the American Heart Association, Circulation 125 (1), pp. 188-97 (January 2012).

In contrast, ischemic heart disease is becoming a more common cause of death in the developing world. For example in India, ischemic heart disease had become the leading cause of death by 2004; accounting for 1.46 million deaths (14% of total deaths). Deaths in India due to ischemic heart disease were also expected to double during 1985-2015. Gupta, et al., Epidemiology and Causation of Coronary Heart Disease and Stroke in India, Heart 94 (1), pp. 16-26 (January 2008).

Globally, it is predicted that disability adjusted life years (DALYs) lost to ischemic heart disease will account for 5.5% of total DALYs in 2030, making it the second most important cause of disability (after unipolar depressive disorder), as well as the leading cause of death by this date.

A myocardial infarction (a common presentation of ischemic heart disease) often occurs when a coronary artery becomes occluded and can no longer supply blood to the myocardial tissue, thereby resulting in myocardial cell death. When a myocardial infarction occurs, the myocardial tissue that is no longer receiving adequate blood flow ultimately dies (without effective intervention) and is eventually replaced by scar tissue.

Within seconds of a myocardial infarction, the under-perfused myocardial cells no longer contract, leading to abnormal wall motion, high wall stresses within and surrounding the infarct, and depressed ventricular function. The high stresses at the junction between the infarcted tissue and the normal tissue lead to expansion of the infarcted area and remodeling, i.e. a cascading sequence of myocellular events, over time.

Various methods for treating a myocardial infarction are often employed. Such methods include stabilizing the hemodynamics associated with a myocardial infarction via systemic delivery of various pharmacological agents and restoring the patency of occluded vessels via thrombolytic therapy or angioplasty and stents.

Several additional methods for treating a myocardial infarction are directed to re-establishing blood flow to the ischemic area through stimulation of angiogenesis. Re-establishing blood flow at the ischemic area can, and in many instances will, reduce symptoms associated with a myocardial infarction and/or improve cardiac function.

Some methods for re-establishing blood flow and rehabilitating the heart involve invasive surgery, such as bypass surgery or angioplasty. Other methods employ lasers to bore holes through the infarctions and ischemic area(s) to promote blood flow. As one can readily appreciate, there are numerous incumbent risks associated with the noted methods.

A further method for treating a myocardial infarction is the direct or selective delivery of bioactive or pharmacological agents to the infarction and/or ischemic area (i.e. effected or damaged cardiovascular tissue). Direct delivery of a bioactive or pharmacological agent to the effected cardiovascular tissue is often preferred over the systemic delivery for several reasons. A primary reason is that a substantially greater concentration of such agents that can be delivered directly into the effected cardiovascular tissue, compared with the dilute concentrations possible through systemic delivery. Another reason is the risk of systemic toxicity which can, and in many instances will, occur with doses of pharmacological agents that are typically required to achieve desired drug concentrations in the effected cardiovascular tissue.

One common method of delivering bioactive or pharmacological agents to effected cardiovascular tissue, e.g. damaged myocardial tissue, comprises advancing a catheter through the vasculature and into the heart to inject the agents directly into the effected cardiovascular tissue from within the heart.

Another method of delivering bioactive or pharmacological agents to effected cardiovascular tissue comprises epicardial, direct injection into the tissue during an open chest procedure.

There are, however, several drawbacks and disadvantages associated with the noted injection methods. One major drawback is that, in many instances, the depth of the injection and delivery pattern of the injected agent is solely dependent upon the surgeon.

It would thus be desirable to provide to provide improved methods and systems for delivering pharmacological compositions directly to damaged or diseased biological tissue; particularly, cardiovascular tissue.

It is therefore an object of the present invention to provide improved methods and systems for delivering pharmacological compositions directly to damaged or diseased biological tissue; particularly, cardiovascular tissue, that overcome the drawbacks and disadvantages associated with prior art methods and systems for delivering pharmacological agents and compositions to biological tissue.

### SUMMARY OF THE INVENTION

The present invention is directed to methods and systems for delivering pharmacological compositions directly to damaged or diseased biological tissue; particularly, cardiovascular tissue. In some embodiments, the delivery system comprises a multi-needle injection system having (i) pneumatic drive system, (ii) a plurality of reservoirs that are configured to receive a pharmacological composition therein and (iii) a needle array having a plurality of needles associated therewith.

In a preferred embodiment of the invention, the method of delivering a pharmacological composition to biological tissue comprises direct delivery or administration of at least one pharmacological composition of the invention to target (e.g. damaged or diseased) biological tissue.

In a preferred embodiment, the pharmacological compositions comprise extracellular matrix (ECM) compositions that include at least one ECM material.

According to the invention, the ECM material can be derived from various mammalian tissue sources, including the small intestine, large intestine, stomach, lung, liver, kidney, mesothelium, pancreas, placenta, heart, bladder, prostate, tissue surrounding growing enamel, tissue surrounding growing bone, and any fetal tissue from any mammalian organ, and methods for preparing same.

In some embodiments, the ECM compositions further include one or more additional biologically active components to facilitate the treatment of damaged tissue and/or the tissue regenerative process.

In some embodiments, the ECM compositions thus include at least one pharmacological agent or composition, which can comprise, without limitation, antibiotics or antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

In some embodiments of the invention, the pharmacological agent specifically comprises an anti-inflammatory agent or composition.

In some embodiments of the invention, the pharmacological agent comprises a statin. According to the invention, suitable statins include, without limitation, atorvastatin, cerivastatin, fluvastatin, lovastatin, mevastatin, pitavastatin, pravastatin, rosuvastatin, and simvastatin.

In some embodiments of the invention, the EMC compositions include chitosan.

In some embodiments of the invention, the EMC compositions include a cell.

In some embodiments of the invention, the ECM compositions include a protein.

In some embodiments of the invention, the ECM compositions are formulated to facilitate injection of the ECM compositions to damaged or diseased tissue (i.e. injectable ECM compositions).

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of one embodiment of a multi-needle injection system, in accordance with the invention;

FIGS. 2A and 2B are perspective views of one embodiment of multi-needle injector apparatus, in accordance with the invention;

FIG. 3 is a further perspective view of the multi-needle injector apparatus shown in FIGS. 2A and 2B, in accordance with the invention;

FIG. 4A is an exploded perspective view of the multi-needle injector apparatus shown in FIGS. 2A and 2B, in accordance with the invention;

FIG. 4B is a perspective view of the front portion of the multi-needle injector apparatus shown in FIGS. 2A and 2B, in accordance with the invention;

FIG. 5A is a perspective view of a needle and associated mounting member, in accordance with the invention;

FIG. 5B is a partial side plan view of the injector apparatus proximal portion showing the needle and associated mounting member shown in FIG. 5A, in accordance with the invention;

FIG. 6 is an illustration of one embodiment of a control system touch screen, in accordance with the invention;

FIG. 7 is a side plan sectional view of one embodiment of an injector needle, in accordance with the invention;

FIG. 8 is a side plan sectional view of another embodiment of an injector needle, in accordance with the invention;

FIG. 9 is a schematic illustration of one embodiment of a multi-needle injector having integral depth control means associated therewith, in accordance with the invention; and

FIG. 10 is a schematic illustration of one embodiment of a control system having depth control means associated therewith, in accordance with the invention.

### DETAILED DESCRIPTION OF THE PREFERRED EMBODIMENT

Before describing the present invention in detail, it is to be understood that this invention is not limited to particularly exemplified apparatus, systems, compositions or methods as such may, of course, vary. Thus, although a number of systems, compositions and methods similar or equivalent to those described herein can be used in the practice of the present invention, the preferred systems, compositions and methods are described herein.

It is also to be understood that, although the systems, pharmacological compositions and methods of the invention are illustrated and described in connection with administration (or delivery) of pharmacological compositions (and bioactive and pharmacological agents) to cardiovascular tissue, the systems, compositions and methods of the invention are not limited to such delivery. According to the invention, the systems and methods of the invention can be employed to administer pharmacological compositions (and bioactive and pharmacological agents) to numerous additional biological tissue, including, without limitation, gastrointestinal and respiratory organ tissue.

It is further to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an anti-inflammatory” includes two or more such agents and the like.

### DEFINITIONS

The terms “cardiac tissue damage”, “cardiac tissue injury” and “cardiovascular tissue damage” are used interchangeably herein, and mean and include any area of abnormal tissue in the cardiovascular system or heart caused by a disease, disorder, injury or damage, including damage to the epicardium, endocardium and/or myocardium. Non-limiting examples of causes of cardiovascular tissue damage include acute or chronic stress (systemic hypertension, pulmonary hypertension, valve dysfunction, etc.), coronary artery disease, ischemia or infarction, inflammatory disease and cardiomyopathies.

As is well known in the art, cardiovascular tissue damage most often involves damage or injury to the myocardium and, therefore, for the purposes of this disclosure, myocardial damage or injury is equivalent to cardiovascular tissue damage.

The term “damaged tissue”, as used herein, means and includes biological tissue; particularly, cardiovascular tissue damaged or injured by trauma, ischemic tissue, infarcted tissue or tissue damaged by any means which results in interruption of normal blood flow to the tissue.

The terms “prevent” and “preventing” are used interchangeably herein, and mean and include reducing the frequency or severity of a disease, condition or disorder. The term does not require an absolute preclusion of the disease, condition or disorder. Rather, this term includes decreasing the chance for disease occurrence.

The terms “treat” and “treatment” are used interchangeably herein, and mean and include medical management of a patient with the intent to cure, ameliorate, stabilize, or prevent a disease, pathological condition or disorder. The terms include “active treatment”, i.e. treatment directed specifically toward the improvement of a disease, pathological condition or disorder, and “causal treatment”, i.e. treatment directed toward removal of the cause of the associated disease, pathological condition or disorder.

The terms “treat” and “treatment” further include “palliative treatment”, i.e. treatment designed for the relief of symptoms rather than the curing of the disease, pathological condition or disorder, “preventative treatment”, i.e. treatment directed to minimizing or partially or completely inhibiting the development of the associated disease, pathological condition or disorder, and “supportive treatment”, i.e. treatment employed to supplement another specific therapy directed toward the improvement of the associated disease, pathological condition or disorder.

The term “chamber remodeling”, as used herein, means and includes a series of events (which may include changes in gene expression, molecular, cellular and interstitial changes) that result in changes in size, shape and function of cardiac tissue following stress or injury. As is well known in the art, remodeling can occur after a myocardial infarction, pressure overload (e.g., aortic stenosis, hypertension), volume overload (e.g., valvular regurgitation), inflammatory heart disease (e.g., myocarditis), or in idiopathic cases (e.g., idiopathic dilated cardiomyopathy).

The term “angiogenesis”, as used herein, means a physiologic process involving the growth of new blood vessels from pre-existing blood vessels.

The term “neovascularization”, as used herein, means and includes the formation of functional vascular networks that can be perfused by blood or blood components. Neovascularization includes angiogenesis, budding angiogenesis, intussuceptive angiogenesis, sprouting angiogenesis, therapeutic angiogenesis and vasculogenesis.

The terms “extracellular matrix”, “extracellular matrix material” and “ECM material” are used interchangeably herein, and mean a collagen-rich substance that is found in between cells in animal tissue and serves as a structural element in tissues. It typically comprises a complex mixture of polysaccharides and proteins secreted by cells. The extracellular matrix can be isolated and treated in a variety of ways. Extracellular matrix material (ECM) can be isolated from small intestine submucosa, stomach submucosa, urinary bladder submucosa, tissue mucosa, dura mater, liver basement membrane, pericardium or other tissues. Following isolation and treatment, it is commonly referred to as extracellular matrix or ECM material.

The terms “pharmacological agent”, “pharmaceutical agent”, “agent”, “active agent”, “drug” and “active agent formulation” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent”, “pharmaceutical agent”, “agent”, “active agent”, “drug” and “active agent formulation” thus mean and include, without limitation, antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

The terms “anti-inflammatory” and “anti-inflammatory agent” are also used interchangeably herein, and mean and include a “pharmacological agent” and/or “active agent formulation”, which, when a therapeutically effective amount is administered to a subject, prevents or treats bodily tissue inflammation i.e. the protective tissue response to injury or destruction of tissues, which serves to destroy, dilute, or wall off both the injurious agent and the injured tissues. Anti-inflammatory agents thus include, without limitation, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

The terms “active agent formulation”, “pharmacological agent formulation” and “agent formulation”, are also used interchangeably herein, and mean and include an active agent optionally in combination with one or more pharmaceutically acceptable carriers and/or additional inert ingredients. According to the invention, the formulations can be either in solution or in suspension in the carrier.

The term “pharmacological composition”, as used herein means and includes a composition comprising a “pharmacological agent” and/or an “extracellular matrix material” and/or a “pharmacological agent formulation.”

The term “therapeutically effective”, as used herein, means that the amount of the “pharmacological composition” and/or “pharmacological agent” and/or “active agent formulation” administered is of sufficient quantity to ameliorate one or more causes, symptoms, or sequelae of a disease or disorder. Such amelioration only requires a reduction or alteration, not necessarily elimination, of the cause, symptom, or sequelae of a disease or disorder.

The terms “ delivery” and “administration” are used interchangeably herein, and mean and include injection of a “pharmacological composition” or “pharmacological agent” or “active agent formulation” to a treatment site, i.e. biological tissue.

The term “percutaneous”, as used herein, means and includes any penetration through the skin of a patient or subject, whether in the form of a small cut, incision, hole, cannula, tubular access sleeve or port or the like.

The terms “patient” and “subject” are used interchangeably herein, and mean and include warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “comprise” and variations of the term, such as “comprising” and “comprises,” means “including, but not limited to” and is not intended to exclude, for example, other additives, components, integers or steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

As will readily be appreciated by one having ordinary skill in the art, the present invention substantially reduces or eliminates the disadvantages and drawbacks associated with prior art methods of delivering pharmacological agents and compositions to biological tissue.

In overview, the present disclosure is directed to methods and systems for delivering pharmacological compositions directly to damaged or diseased biological tissue; particularly, cardiovascular tissue. The present invention is also directed to methods and systems for treating damaged and diseased biological tissue; particularly, cardiovascular tissue, via the “direct” delivery of a pharmacological composition (and/or pharmacological agent and/or formulation) to the damaged or diseased tissue. According to the invention, the delivery of a therapeutically effective amount of a pharmacological composition of the invention to damaged or diseased tissue induces neovascularization, host tissue proliferation, bioremodeling and regeneration of new tissue.

### Injection Delivery Systems

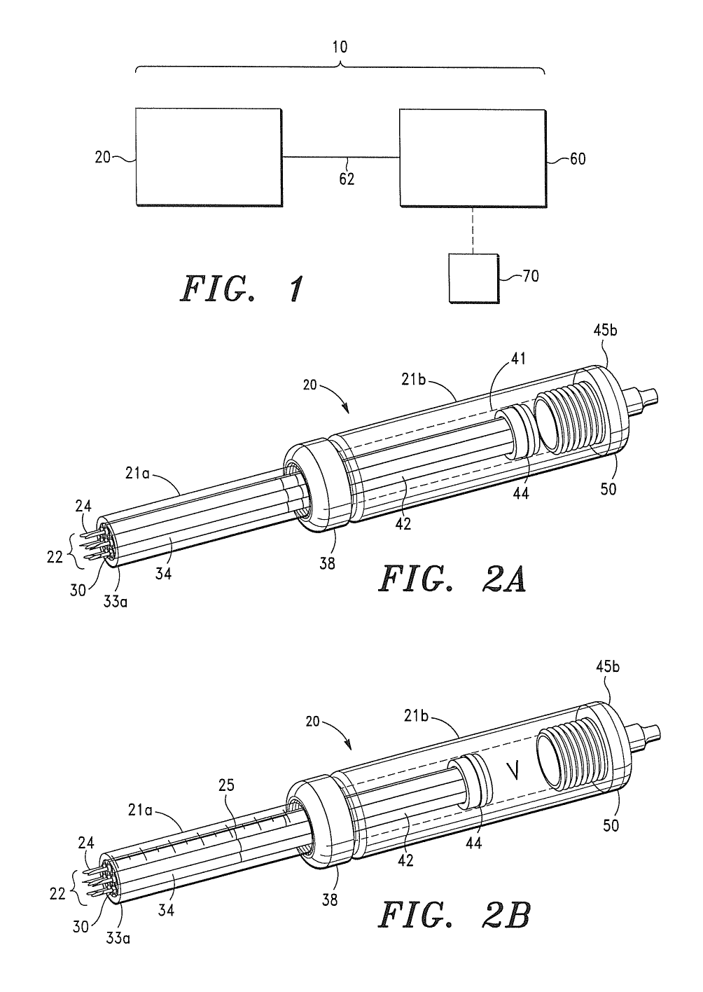

Referring now to FIGS. 1-10, a preferred embodiment of a multi-needle injection system of the invention will be described in detail. It is, however, understood that although the illustrated and described multi-needle injection system employs a pneumatic actuation means, i.e. driving force, the system is not limited to pneumatic actuation means. Indeed, according to the invention, the multi-needle injection systems of the invention can also employ hydraulic and mechanical actuation means.

Referring first to FIG. 1, there is shown a schematic illustration of the multi-needle injection system 10. As illustrated in FIG. 1, the system 10 includes an injector apparatus 20 and associated control system 60.

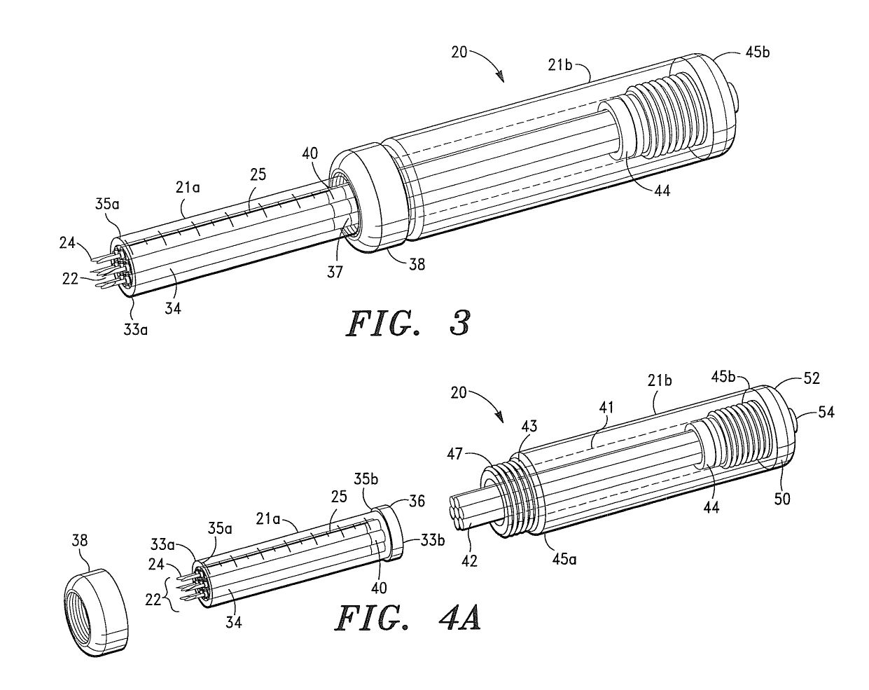

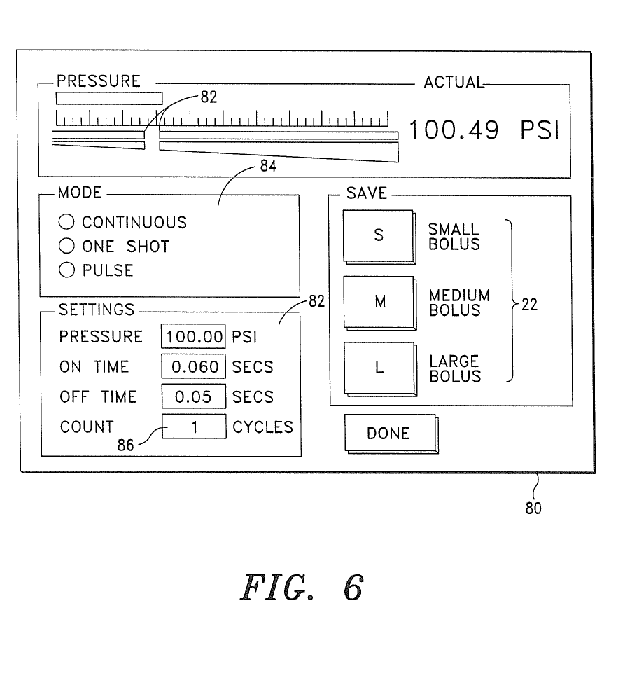

Referring now to FIGS. 2A, 2B and 3, in preferred embodiment of the invention, the injection apparatus 20 includes (i) a first housing 21a having a needle array 22, a needle mounting member 30 (see FIG. 5) and a plurality of reservoirs 34 contained therein, and (ii) a second housing 21b having a plurality of reservoir piston drive members 42, a central drive piston 44 and driving force receiving means 50.

In some embodiments of the invention, the first housing 21a preferably has an outer diameter in the range of approximately 1-50 mm, more preferably, in the range of approximately 10-25 mm. It is contemplated that the diameter of the first housing 21a is a function of the desired dosage to be delivered to the target tissue area, the size of the needle array 22 and the size of each individual needle 24.

In some embodiments of the invention, the second housing 21b preferably has an outer diameter in the range of approximately 2-51 mm, more preferably, in the range of approximately 11-26 mm. It is similarly contemplated that the diameter of the second housing 21b is a function of the desired dosage to be delivered to the target tissue area, the size of the needle array 22 and the size of each reservoir 34 internal lumen diameter.

In a preferred embodiment, each needle mounting member 30 is securely engaged to the first end 33a (i.e. proximal end) of the first housing 21a (see FIG. 5B). In some embodiments, each needle mounting member 30 is an integral component of the first housing 21a (e.g. formed as a single unit).

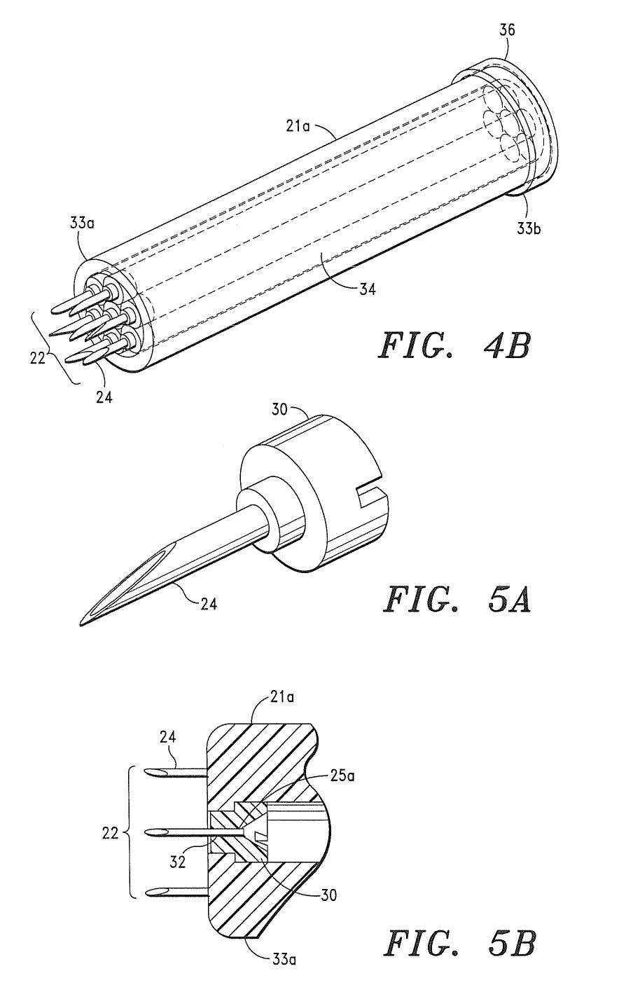

As illustrated in FIG. 4A and 4B, the first housing 21a preferably includes a flanged region 36 on a distal end 33b thereof that is configured to be received by a housing seat 43 on a first end 45a of the second housing 21b. The first end 45a of the second housing 21b further includes a plurality of threads 47 that are configured to threadably receive (and cooperate with) internal threads of the housing retainer 38, whereby the housing retainer 38 securely engages the first 21a and second 21b housings together when the housing retainer 38 is engaged to (and tightened on) the second housing threads 47.

As illustrated in FIGS. 2A-5B, the needle array 22 comprises a plurality of needles 24. In a preferred embodiment of the invention, each needle 24 includes an internal lumen 26 (see FIG. 7), having, in the illustrated embodiment, a nominal diameter in the range of 0.159-1.194 mm (i.e. 16 g-30 g), extending substantially the entire length of the needle 24.

According to the invention, the length of each needle 24 can be in the range of 2-25 mm. Thus, in some embodiments of the invention, the length of each needle 24 is in the range of approximately 3-10 mm. In some embodiments, the length of each needle 24 is in the range of approximately 4-5 mm.

It is contemplated that the length of each needle 24 is a function of the depth of desired dosage delivery. It is further contemplated that the length of each needle 24 is dependent upon the needle point angle α, as shown in FIG. 7.

In some embodiments of the invention, at least one, more preferably, each needle 24 is coated with an immunomodulating compound.

In some embodiments, the immunomodulating compound comprises a polysaccharide, including, without limitation, GAGs, dextrans, alginate and chitosan.

In some embodiments, immunomodulating compound comprises a polymeric material, including, without limitation, high molecular weight hyaluronic acid (HMW-HA).

According to the invention, the needle array 22 can comprise any number of needles 24, e.g., two (2), six (6), seven (7), nine (9), twelve (12), etc. In the embodiment shown in FIGS. 2A and 2B, the needle array 22 comprises seven (7) needles 24.

According to the invention, the needle array 22 can also comprise various patterns. As shown in FIG. 4B, in some embodiments, the needles 24 are substantially equally spaced within a substantially circular pattern.

According to the invention, the circular pattern can comprise various diameters. In one embodiment, the circular pattern has a diameter in the range of 0.40-0.50 in.

In some embodiments of the invention, the needles 24 are substantially spaced and arranged in linear patterns, e.g. substantially parallel linear patters, crossing linear patterns, etc.

According to the invention, the needles 24 can comprise various materials, including, without limitation, stainless steel, nitinol, nichrome, MP35N, and elgiloy. In a preferred embodiment of the invention each needle 24 comprises a biocompatible material. Suitable biocompatible materials include, without limitation, magnesium, and PEEK™.

Referring now to FIG. 5B, each needle 24 is coupled, at a first end 25a, in a respective needle aperture 32 extending in the needle mounting member 30.

Each needle 24 is further in communication with an associated (or dedicated) reservoir 34 proximate a first end 35a thereof. Each reservoir 34 has an internal lumen that is designed and configured to receive a pharmacological composition therein.

In some embodiments of the invention, each reservoir 34 internal lumen has a diameter in the range of 0.1-10 mm. In some embodiments, each reservoir 34 internal lumen has a diameter in the range of 0.4-0.6 mm.

According to the invention, each reservoir 34 can be configured to receive and contain a predetermined amount of a pharmacological composition therein. Preferably, each reservoir 34 is designed and adapted to receive and contain at least 0.05 cc of a pharmacological composition therein.

It is contemplated that the amount of a pharmacological composition that each reservoir 34 is designed and adapted to receive is a function of the number of needles 24, and the desired overall dosage.

Thus, in some embodiments of the invention, each reservoir 34 is designed and adapted to receive and contain in the range of approximately 0.01-1.5 cc of a pharmacological composition therein. In some embodiments, each reservoir 34 is designed and adapted to receive in the range of approximately 0.05-1.0 cc of a pharmacological composition therein. In some embodiments, each reservoir 34 is designed and adapted to receive in the range of approximately 0.1-0.2 cc of a pharmacological composition therein.

According to the invention, each reservoir 34 can similarly comprise various materials, including, without limitation, acrylic, polycarbonate, PTFE and ABS.

Each reservoir 34 is further adapted to slidably receive a reservoir piston 40 in the distal end 35b of the reservoir 34. According to the invention, the reservoir piston 40 can comprise various materials and shapes, including substantially cylindrical (as shown in FIGS. 2A and 2B) and circular. Suitable materials comprise, without limitation, Teflon® (PTFE), ultra-high-molecular-weight-polyethylene (UHMWPE), perfluoroalkoxy (PFA), and other like materials.

Referring now to FIGS. 2A and 2B, the second housing 21b of the injector apparatus 20 includes a plurality of reservoir piston drive members or rods 42, a central drive piston 44, and driving force receiving means 50. As illustrated in FIG. 2A, the second housing 21b includes an internal lumen 41 that is configured to receive the reservoir piston drive members 42 and central drive piston 44 therein.

According to the invention, the diameter and, hence, internal volume of the second housing 21b internal lumen 41 can vary, dependent upon the desired pressure to be applied to the reservoir pistons 40.

In some embodiments of the invention, the second housing 21b internal lumen 41 has a diameter in the range of approximately 1-50 mm. In some embodiments, the second housing 21b internal lumen 41 has a diameter in the range of approximately 10-25 mm.

It is contemplated that the size of the central drive piston 44 is a function of the desired output pressure to each reservoir piston 40. In a non-limiting example, the desired ratio of the area of the central drive piston 44 to each reservoir piston 40 is at least 0.5:1. More preferably, the desired ratio of the area of the central drive piston 44 to each reservoir piston 40 is 1:1, whereby the pressure that is applied to the central drive piston 44 is substantially equivalent to each pressure applied individually to each reservoir piston 40.

In some embodiments of the invention, each reservoir piston drive member 42 has a reservoir mating (i.e. outer) diameter that is at least 50 μm smaller than the reservoir 34 internal lumen diameter.

Thus, in some embodiments of the invention, each reservoir piston drive member 42 has an outer diameter in the range of approximately 0.050-9.5 mm. In some embodiments, each reservoir piston drive member 42 has an outer diameter in the range of approximately 0.350-0.550 mm.

According to the invention, the length of each reservoir piston drive member 42 can vary based upon several factors, such as the range of desired delivery volume of a pharmacological composition, the capacity of each needle relative to the desire dose and the impact of leakage (or “creep”). In the illustrated embodiment, the length of each reservoir piston drive member 42 is in the range of approximately 75-125 mm.

As illustrated in FIGS. 2A and 2B, each reservoir piston drive member 42 is in communication with the central drive piston 44. In some embodiments, such as shown in FIGS. 2A and 2B, each reservoir piston drive member 42 is securely engaged to the central drive piston 44.

In some embodiments, the central drive piston 44 has an outer diameter that is at least 0.050 mm smaller than the second housing 21b internal lumen 41. In some embodiments of the invention, the central drive piston 44 has an outer diameter that is at least in the range of approximately 1.0-3.0 mm smaller than the second housing 21b internal lumen 41.

In a preferred embodiment of the invention, the central drive piston 44 is sealably received by (and, hence, in) the second housing 21b internal lumen 41.

In the illustrated embodiment, the driving force receiving means 50 includes means for sealing the second housing 21b internal lumen 41, i.e. an end cap 52, and, in the illustrated embodiment, means for receiving a pneumatic driving force 54 from the control system 60.

In pneumatic driven actuators, such as illustrated in FIGS. 2A and 2B, the driving force receiving means 54 comprises a pneumatic fitting that is configured to receive an air line 62 that is in communication with the control system 60 (see FIG. 10).

In the illustrated embodiment, the control system 60 includes actuation control means 70 (see FIG. 1), display means (discussed in detail below), and, in the illustrated embodiment, a source of compressed air to provide the pneumatic driving force(s) for the injector apparatus 20.

In a preferred embodiment of the invention, the control system is programmed and configured to regulate at least the pneumatic driving or actuation force(s) and, thereby, delivery of the pharmacological composition. Preferably, such regulation includes, without limitation, the actuation pressure and mode of delivery, e.g. continuous, single pulse, multiple pulses, frequency of pulses, bolus, etc.

According to the invention, the pressure provided by the control system 60 is at least 5 psi. In some embodiments, the actuation pressure is preferably in the range of approximately 5-1000 psi.

In some embodiments, the actuation pressure is preferably in the range of approximately 100-175 psi. In a preferred embodiment, the actuation pressure is in the range of approximately 140-160 psi.

As indicated, in some embodiments, the mode of actuation pressure can comprise continuous, single pulse, multiple pulses. According to the invention, the number of pulses can comprise 2-1000 pulses. In a preferred embodiment, the number of pulses is in the range of approximately 1-10 pulses.

According to the invention, the frequency of the pulses can range from 0.1 Hz-5 kHz. In a preferred embodiment, the frequency of the pulses is in the range of 1 kHz-2 kHz.

According to the invention, the duty cycle of the pulses can range from 0.01%-99.99%. In a preferred embodiment, the duty cycle of the pulses is in the range of 33.3% -50.0%.

In a preferred embodiment, the control system 60 is designed and configured to perform at least one or more of the following functions: (i) regulate the actuation pressure, regulate the mode of delivery of the actuation pressure and, hence, delivery of the pharmacological composition, regulate the frequency of pulsed actuation pressure delivery, regulate the bolus, and regulate the number of agent deliveries that occur if the delivery is to be continuous.

In some embodiments, the control system 60 is further designed to monitor various system parameters status, including, without limitation, pneumatic pressure availability, and the electronic and electro-mechanical power sources.

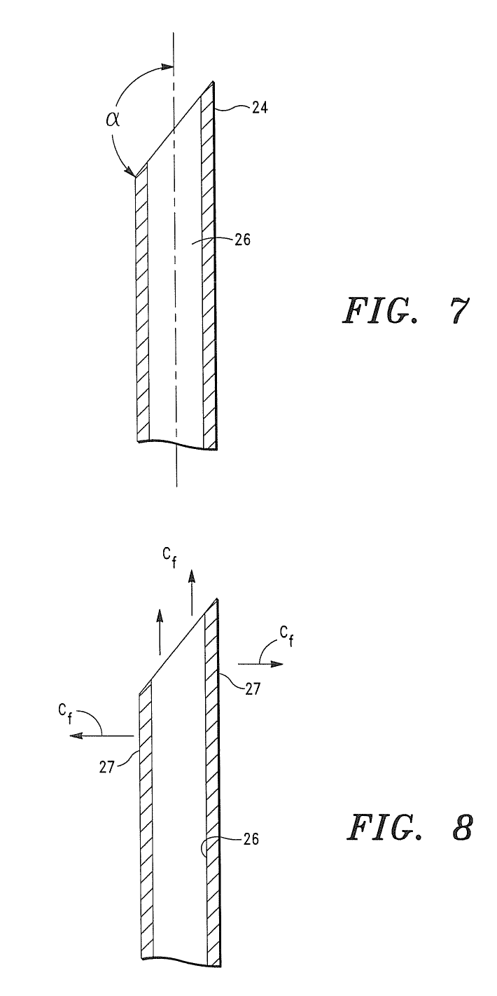

As indicated above, in a preferred embodiment of the invention, the control system 60 also includes display means. In some embodiments of the invention, the display means comprises a touch screen display, such as shown in FIG. 6.

In the embodiment illustrated in FIG. 6, the screen display 80 provides input means and visual indications of actuation pressure 82, mode 84 and frequency 86. The screen display 80 also provides input means and a visual indication of three (3) predetermined (or programmed) bolus selections 88.

According to the invention, the screen display can also provide numerous additional inputs and visual indications, including, without limitation, the pressure available in the pressure supply. In alternative embodiments, wherein a sensing mechanism is used in conjunction with the injector apparatus, such as discussed below, the display can show a representation of the sensing mechanism data.

The touch screen display 80 can also include multiple windows and modes. In some embodiments, the touch screen display further includes a research setting and a physician setting. In some embodiments, the research setting allows the operator, i.e. surgeon, to adjust the frequency, duty cycle, cycle number, and bolus size.

In some embodiments, the physician setting only allows the operator to adjust the bolus size.

In a further envisioned embodiment, there is also an option to switch between displays or screens, wherein a bolus size setting can be adjusted in the research setting, and subsequently switch the display to the physician setting to test the new settings for that respective bolus size.

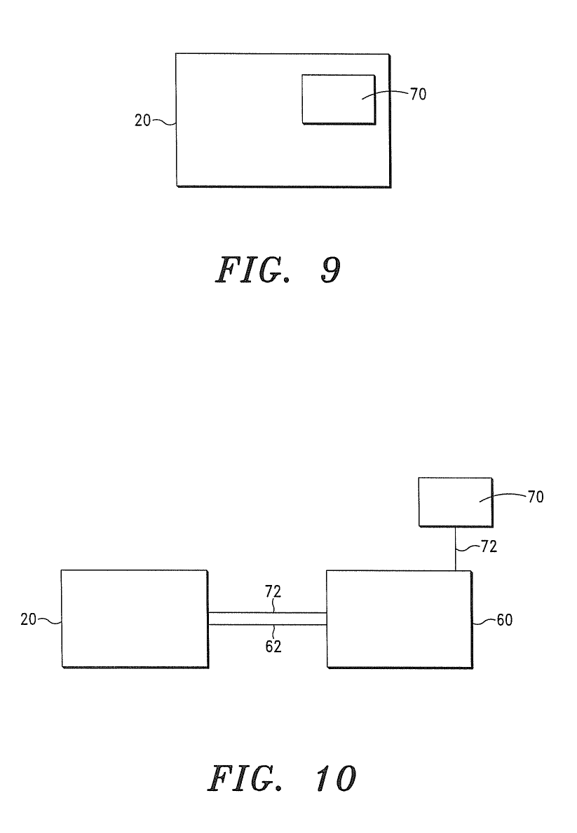

As indicated above, in a preferred embodiment, the control system 60 further includes actuation control means 70. In some embodiments, the actuation control means comprises a foot pedal that is easily accessible by a surgeon or clinician. In some envisioned embodiments, the actuation control means 70 is incorporated in, i.e. an integral component, of the injector actuator 20 (see FIG. 9).

In a preferred embodiment, the actuation control means 70 receives a value from the foot pedal (in some embodiments via a digital to analog converter), and, thereby, determines the foot pedal position. In alternative embodiments, control means data, such as speed and force of foot pedal activation, is used to alter the injection actuator function, such as for example, injection speed or frequency.

In some embodiments of the invention, the injector control system 60 includes energy loss compensation means that is programmed to maintain substantially equal pneumatic delivery pressure to the reservoir piston 40 during programmed delivery of a pharmacological composition. In some embodiments, the energy loss compensation means comprises programmed variable driving pressure, e.g. as the volume of the second housing 21b internal lumen 41 (denoted “V” in FIG. 2B) increases (as the piston 44 is driven forward), the drive pressure is increased by a predetermined amount.

In some embodiments of the invention, the multi-needle injection system 10 includes delivery volume control means. In some embodiments, the delivery volume control means comprises gradations 25 on the injector first housing 21a (see FIG. 3) to provide a visual indication of the volume of a pharmacological composition delivered out of each reservoir 34 and, hence, needle 24 and into target tissue.

In some embodiments of the invention, the volume control means comprises one or more volume sensors, e.g., MEMs sensor, that determine volume of the contained pharmacological composition discharged from one or more reservoirs 34. In some embodiments, the MEMs sensor(s) are disposed within the needle mounting member 30 and in communication with at least one needle aperture 32.

In some embodiments of the invention, the multi-needle injection system 10 also includes delivery pattern control means, i.e. means for controlling the dispersal pattern of pharmacological compositions within tissue. In some embodiments, the delivery pattern control means comprises or is achieved by defined angles of the needle output lumens, i.e. tips. Thus, in some embodiments, at least one needle 24 has a tip angle α (i.e. the angle relative to the longitudinal axis of the needle 24) in the range of approximately 45°-89°, more preferably, in the range of approximately 60°-89° (see FIG. 7).

Referring now to FIG. 8, in some embodiments of the invention, at least one needle 24, more preferably, each needle 24, has a plurality of additional output lumens 27 proximate the needle tip, whereby a pharmacological composition can flow out of each needle 24 as denoted by arrows C_{f}.

In some embodiments of the invention, the multi-needle injection system 10 further includes needle and, hence, injection depth control means. In one embodiment, the injection depth control means comprises a spacer positioned over each needle shaft and removably secured on the first housing 21a end, at a desired distance from the needle distal tips, to prevent penetration into tissue beyond a specified depth. In some embodiments, the spacer is securely attached to the first housing 21a end.

In some embodiments, the injection depth control means comprises one or more ultrasound systems. In some embodiments, the injection depth control means includes a first ultrasound system that is designed and configured to determine the thickness of the tissue to be injected.

In some embodiments, the display means is programmed and configured to display tissue thickness data. In alternative embodiments, the display means is programmed and configured to display a visual representation of the tissue.

In some embodiments, the injection depth control means includes a second ultrasound system that is designed and configured to provide visual representations of delivery volume and agent dispersement after injection.

According to the invention, one means of achieving this particular data acquisition comprises subtracting the secondary thickness data from the initial measurement of tissue thickness, thus showing the shape and location of the injected material.

According to the invention, one or both of the noted ultrasound systems can be an integral component or subsystem of the injector apparatus 20 or the control system 60. One or both of the noted ultrasound systems can alternatively be a separate component or subsystem that is in communication with the injector apparatus 20 and/or control system 60 (via line 72), as shown in FIG. 10.

In some embodiments of the invention, the injection depth control means comprises a CO₂ pressure sensing system. In the noted embodiment, a central CO₂ needle is provided within or proximate the injection needle array 22. The central CO₂ needle is in communication with a CO₂ source and CO₂ pressure regulating means. According to the invention, the CO₂ pressure regulating means is designed and configured to provide CO₂ gas into and through the CO₂ needle and determine reductions in pressure as the CO₂ needle penetrates biological tissue. Thus, as the CO₂ needle is disposed in and traversing through biological tissue, a first pressure will be reflected. When the CO₂ needle enters into a body lumen, e.g. cardiovascular vessel, a second, reduced pressure will be reflected.

In some embodiments of the invention, the injection depth control means comprises a light system. In some embodiments, the light system comprises optical coherence tomography (OCT).

In some embodiments, the data obtained via OCT can be presented on the display means such that the data is numerical or pictorial to allow the surgeon better understanding of the tissue to be injected, as well as the location and orientation of the injected material. In some embodiments, the OCT system is integrally attached to the injection system or, in alternative embodiments, comprises a tangentially connected sub-system that delivers data to the injection system.

If a surgeon or clinician is practicing the current invention using a minimally invasive or percutaneous technique, he/she may also need or require real-time visualization or navigation to ensure site-specific injection. Thus, in some embodiments of the invention, the multi-needle injection apparatus 20 and/or system 60 includes or employs MNav technologies to superimpose pre-operative MRI or CT images onto images of a delivery apparatus to track it in real-time to target sites.

In one embodiment, the surgeon or clinician employs a contrast agent and/or navigation technologies to track the one or more needle needles 24 during injection in a virtual 3-D environment.

The needle array 22 or assembly (or other apparatus component) can further include a feedback element or physiological sensor for measuring a physiological condition to guide delivery of pharmacological compositions to the desired location. For example, an EKG lead may be included on the first housing 21a end or on the distal tip of a needle 24 or otherwise delivered within the selected tissue region to detect and guide injection towards electrically silent or quiet areas of cardiac tissue, or to allow electrical events within the heart to be monitored during delivery of the composition. During treatment, for example, the pharmacological composition may be delivered into a tissue region until a desired condition is met. Also, local EKG monitoring can be used to target and guide injection towards electrically silent or quiet areas of cardiac tissue.

In some embodiments of the invention, the multi-needle injection system 10 further includes heating and/or cooling means to regulate the temperature of pharmacological compositions contained in the injector apparatus 20 reservoirs 34.

### Pharmacological Compositions

According to the invention, various pharmacological compositions can be administered or delivered to biological tissue with a multi-needle injection system of the invention. Suitable pharmacological compositions are disclosed in Co-Pending U.S. application Ser. Nos. 11/182,551, 13/732,943, 13/573,569, and 13/782,115, which are incorporated by reference herein in their entirety.

In some embodiments, one or more pharmacological compositions are directly administered to target biological tissue, e.g., damaged or diseased tissue, via a multi-needle injection system of the invention.

In a preferred embodiment, the pharmacological compositions comprise extracellular matrix (ECM) compositions that include at least one extracellular matrix (hereinafter “ECM material”).

According to the invention, the ECM material can be derived from various mammalian tissue sources and methods for preparing same, such as disclosed in U.S. Pat. Nos. 7,550,004, 7,244,444, 6,379,710, 6,358,284, 6,206,931, 5,733,337 and 4,902,508 and U.S. application Ser. No. 12/707,427; which are incorporated by reference herein in their entirety. The mammalian tissue sources include, without limitation, the small intestine, large intestine, stomach, lung, mesothelium, liver, kidney, pancreas, placenta, heart, bladder, prostate, tissue surrounding growing enamel, tissue surrounding growing bone, and any fetal tissue from any mammalian organ.

According to the invention, the ECM material can be used in whole or in part, so that, for example, an ECM material can contain just the basement membrane (or transitional epithelial layer) with the subadjacent tunica propria, the tunica submucosa, tunica muscularis, and tunica serosa. The ECM material component of the composition can contain any or all of these layers, and thus could conceivably contain only the basement membrane portion, excluding the submucosa.

According to the invention, the ECM material can be formed into a particulate and fluidized, as described in U.S. Pat. Nos. 5,275,826, 6,579,538 and 6,933,326, to form an ECM composition of the invention.

According to the invention, various conventional means can be employed to form a particulate ECM material. In some embodiments, the ECM material is formed into a sheet, fluidized (or hydrated), if necessary, frozen and ground.

In some embodiments of the invention, the ground ECM material is subsequently filtered to achieve a desired particulate size. Thus, in some embodiments, the ECM material has a particulate size no greater than 2000 microns. In some embodiments, the ECM material preferably has a particulate size no greater than 500 microns. In a preferred embodiment, the ECM material has a particulate size in the range of about 20 microns to about 300 microns.

According to the invention, fluidized or emulsified compositions (the liquid or semi-solid forms) can comprise various certain concentrations of ECM material. In some embodiments of the invention, the concentration of the ECM material is greater than about 5%, more preferably, greater than about 20%, even more preferably, greater than about 70%.

As indicated above, in some embodiments of the invention, the ECM compositions are formulated to be injected into damaged or cardiovascular tissue, i.e. injectable ECM compositions. In some embodiments of the invention, the injectable ECM compositions thus comprise approximately 70% particulate ECM material and approximately 30% fully hydrolyzed ECM gel.

According to the invention, the pharmacological compositions of the invention can further include one or more additional bioactive agents or components to aid in the treatment of damaged tissue and/or facilitate the tissue regenerative process.

In some embodiments, the pharmacological compositions of the invention thus include at least one pharmacological agent or composition, which can comprise, without limitation, antibiotics or antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

In some embodiments of the invention, the pharmacological agent specifically comprises an anti-inflammatory agent. Suitable anti-inflammatory agents are set forth in Co-Pending U.S. application Ser. No. 13/573,569.

In some embodiments of the invention, the pharmacological agent comprises a statin, i.e. a HMG-CoA reductase inhibitor. According to the invention, suitable statins include, without limitation, atorvastatin (LIPITOR®), cerivastatin, fluvastatin (Lescol®), lovastatin (Mevacor®, Altocor®, Altoprev®), mevastatin, pitavastatin (Livalo®, Pitava®), pravastatin (Pravachol®, Selektine®, Lipostat®), rosuvastatin (Crestor®), and simvastatin (Zocor®, Lipex®). Several actives comprising a combination of a statin and another agent, such as ezetimbe/simvastatin (Vytorin®), are also suitable.

In some embodiments of the invention, the bioactive agent comprises a chitin derivative, such as chitosan.

In some embodiments of the invention, the bioactive agent comprises a cell. According to the invention, the cell can comprise, without limitation, a stem cell, such as, for example, a human embryonic stem cell, fetal cell, fetal cardiomyocyte, myofibroblast, mesenchymal stem cell, autotransplanted expanded cardiomyocyte, adipocyte, totipotent cell, pluripotent cell, blood stem cell, myoblast, adult stem cell, bone marrow cell, mesenchymal cell, embryonic stem cell, parenchymal cell, epithelial cell, endothelial cell, mesothelial cell, fibroblast, myofibroblast, osteoblast, chondrocyte, exogenous cell, endogenous cell, stem cell, hematopoetic stem cell, pluripotent stem cell, bone marrow-derived progenitor cell, progenitor cell, myocardial cell, skeletal cell, undifferentiated cell, multi-potent progenitor cell, unipotent progenitor cell, monocyte, cardiomyocyte, cardiac myoblast, skeletal myoblast, macrophage, capillary endothelial cell, xenogenic cell, and allogenic cell.

In some embodiments of the invention, the bioactive agent comprises a protein. According to the invention, the protein can comprise, without limitation, a growth factor, collagen, proteoglycan, glycosaminoglycan (GAG) chain, glycoprotein, cytokine, cell-surface associated protein, cell adhesion molecule (CAM), angiogenic growth factor, endothelial ligand, matrikine, matrix metalloprotease, cadherin, immunoglobin, fibril collagen, non-fibrillar collagen, basement membrane collagen, multiplexin, small-leucine rich proteoglycan, decorin, biglycan, fibromodulin, keratocan, lumican, epiphycan, heparan sulfate proteoglycan, perlecan, agrin, testican, syndecan, glypican, serglycin, selectin, lectican, aggrecan, versican, nuerocan, brevican, cytoplasmic domain-44 (CD44), macrophage stimulating factor, amyloid precursor protein, heparin, chondroitin sulfate B (dermatan sulfate), chondroitin sulfate A, heparan sulfate, hyaluronic acid, fibronectin (Fn), tenascin, elastin, fibrillin, laminin, nidogen/entactin, fibulin I, fibulin II, integrin, a transmembrane molecule, platelet derived growth factor (PDGF), epidermal growth factor (EGF), transforming growth factor alpha (TGF-alpha), transforming growth factor beta (TGF-beta), fibroblast growth factor-2 (FGF-2) (also called basic fibroblast growth factor (bFGF)), thrombospondin, osteopontin, angiotensin converting enzyme (ACE), and vascular epithelial growth factor (VEGF).

According to the invention, the bioactive agents referenced above can comprise any form. In some embodiments of the invention, the bioactive component or components, e.g. simvastatin and/or chitosan, comprise microcapsules that provide delayed delivery of the agent contained therein.

Additional suitable pharmacological compositions that can be delivered within the scope of the invention are disclosed in Pat. Pub. Nos. 20070014874, 20070014873, 20070014872, 20070014871, 20070014870, 20070014869, and 20070014868; which are expressly incorporated by reference herein in its entirety.

### Delivery to Target Biological Tissue

As indicated above, the multi-injection system of the invention can be employed to deliver one or more pharmacological compositions to various biological organs and/or tissue.

In some embodiments of the invention, one or more ECM compositions of the invention are directly administered or delivered to damaged or diseased cardiovascular tissue via a multi-needle injection apparatus and/or system of the invention. According to the invention, the ECM compositions can be directly administered to the heart wall and/or the various cardiovascular structures associated therewith.

As is well known in the art, the human heart wall consists of an inner layer of simple squamous epithelium, referred to as the endocardium. The endocardium overlays the myocardium (a variably thick heart muscle) and is enveloped within a multi-layer tissue structure referred to as the pericardium. The innermost layer of the pericardium, referred to as the visceral pericardium or epicardium, covers the myocardium. An outermost layer of the pericardium, referred to as the fibrous pericardium, attaches the parietal pericardium to the sternum, the great vessels and the diaphragm.

According to the invention, an ECM composition can be delivered to each of the noted structures; particularly, the myocardium with a multi-needle injection apparatus of the invention, whereby neovascularization, host tissue proliferation, and bioremodeling is induced.

As indicated above, myocardial infarction, i.e. irreversible myocardial injury resulting in necrosis of a significant portion of myocardium, can result in an acute depression in ventricular function and expansion of the infarcted tissue under stress. This triggers a cascading sequence of myocellular events. In many cases, this progressive myocardial infarct expansion and remodeling leads to deterioration in ventricular function and heart failure.

When a myocardial infarction occurs, the myocardial tissue that is no longer receiving adequate blood flow dies and is replaced with scar tissue. This infarcted tissue cannot contract during systole, and may actually undergo lengthening in systole and leads to an immediate depression in ventricular function. This abnormal motion of the infarcted tissue can cause delayed or abnormal conduction of electrical activity to the still surviving peri-infarct tissue (tissue at the junction between the normal tissue and the infarcted tissue) and also places extra structural stress on the peri-infarct tissue.

In addition to immediate hemodynamic effects, the infarcted heart tissue and undergoes three major processes: infarct expansion, infarct extension, and chamber remodeling. These factors individually and in combination contribute to the eventual dysfunction observed in the cardiac tissue remote from the site of the infarction.

Infarct expansion is a fixed, permanent, disproportionate regional thinning and dilatation of tissue within the infarct zone. Infarct extension is additional myocardial necrosis following myocardial infarction. Infarct extension results in an increase in total mass of infarcted tissue.

The noted effects of a myocardial infarction can, however, be ameliorated or eliminated by administering an ECM composition of the invention directly to the infarcted cardiovascular tissue. As set forth in Co-Pending application Ser. Nos. 11/182,551 and 13/573,569, the ECM compositions will induce neovascularization, host tissue proliferation, bioremodeling, and regeneration of new cardiac tissue structures with site-specific structural and functional properties.

### EXAMPLES

The following examples are provided to enable those skilled in the art to more clearly understand and practice the present invention. They should not be considered as limiting the scope of the invention, but merely as being illustrated as representative thereof.

### Example 1

Five (5) porcine hearts were obtained from young calves. After removal, the hearts were stored in a saline bath.

A first heart was removed from the bath. The thickness of the heart wall was determined to range from 4 mm to greater than 2 cm with an A scan ultrasound sensor.

A multi-needle injection system of the invention, such as illustrated in FIGS. 2 and 3 was provided and prepared for the injection procedure.

An ECM composition of the invention was also provided. The ECM composition comprised two components: an ECM (i.e. SIS) particulate derived from porcine intestines and a SIS gel. The SIS particulate comprised SIS material, which was cryogenically ground to a characterized particle size, and subsequently thawed and loaded into a syringe for delivery. The particulate size was in the range of 50-350 microns.

The SIS gel comprised SIS material that was cryogenically ground, subject to enzymatic digestion in acid, lyophilized, and reconstituted to a predetermined concentration. The SIS gel was also subjected to a subsequent disinfection and neutralization process. The SIS gel was also loaded into a syringe.

The materials were maintained in refrigerated conditions throughout processing.

Approximately 4 cc of SIS gel was mixed with 6 cc of particulate SIS to derive an injectable ECM composition.

The injectable ECM composition was then transferred into the reservoirs of the injector apparatus.

The injector control system was then set to provide the following delivery parameters: two (2) equal pulses at 20 and 30 milliseconds and at pressures ranging from approximately 60-120 psi. The noted parameters provided an ECM composition delivery in the range of approximately 0.5-1.0 ml per pulse.

The ECM composition was then delivered into the wall of a first heart. The injected portion of the heart wall was then observed visually and with a B scan ultrasound (i.e. echo) sensor to assess the ECM composition delivery pattern. Substantially uniform delivery (i.e. amount and spread) at each needle injection site was observed.

The injected portion of the heart wall was also sectioned to observe the delivery pattern. The procedure confirmed that delivery was uniform and at the prescribed needle depth at each needle injection site, with a good safety margin from the ventricular cavity.

The above noted test procedures were similarly employed with the remaining four (4) porcine hearts. The only parameter that varied was the proportion of SIS gel in the ECM composition.

In the second heart, the ECM composition was similar to the ECM composition employed for the first heart, i.e. approximately 4 cc of SIS gel and 6 cc of particulate SIS.

In the third and fourth hearts, the ECM composition comprised approximately 2 cc of SIS gel and 8 cc of particulate SIS.

In the fifth heart, no SIS gel was employed. The ECM composition thus comprised approximately 10 cc of particulate SIS.

In each instance, the delivery was similarly uniform and at the prescribed needle depth at each needle injection site, with a good safety margin from the ventricular cavity.

### Example 2

A young porcine was provided in which CHF had been induced via serial microsphere injections down the coronary arteries.

A multi-needle injection system of the invention, such as illustrated in FIGS. 2 and 3, was prepared for injection of an ECM composition of the invention.

An ECM composition, such as described in Example 1, was also provided. The composition mixture comprised approximately 4 cc of SIS gel was mixed with 6 cc of particulate SIS to derive an injectable ECM composition.

The injectable ECM composition was then transferred into the reservoirs of the injector apparatus.

The injector control system was similarly set to provide the following delivery parameters: two (2) equal pulses at 20 and 30 milliseconds and at pressures ranging from approximately 60-120 psi. The noted parameters provided an ECM composition delivery in the range of approximately 0.5-1.0 ml per pulse.

The heart of the porcine was then exposed. A B scan ultrasound sensor was then employed to assess the depth of the infarcted region.

The ECM composition was then delivered into the infarcted region. The injected portion of the heart wall was then observed visually and with a B scan ultrasound (i.e. echo) sensor to assess the ECM composition delivery pattern. Substantially uniform delivery (i.e. amount and spread) at each needle injection site was observed. The ECM composition also stayed within the infarcted region without coming out of the ventricle wall.

Further, no extravisation or emboli was observed.

A ventricular assist device was subsequently placed into the apex and the animal recovered without incident.

In accord with the invention, within 2-4 weeks, neovascularization, host tissue proliferation, bioremodeling and regeneration of new tissue proximate the infracted region will be observed.

As will readily be appreciated by one having ordinary skill in the art, the present invention provides numerous advantages compared to prior art methods and systems for treating damaged cardiac tissue. Among the advantages are the following:

The provision of pharmacological compositions which, when delivered to damaged biological tissue; particularly, cardiovascular tissue, induce neovascularization, and promote survival and regeneration of damaged cardiovascular tissue.

The provision of extracellular matrix (ECM) compositions which, when delivered to damaged biological tissue; particularly, cardiovascular tissue, induce host tissue proliferation, bioremodeling, and regeneration of cardiovascular tissue structures with site-specific structural and functional properties.

The provision of improved methods and systems for administering pharmacological compositions; particularly, ECM compositions directly to damaged or diseased biological tissue.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. An injector system for delivery of a pharmacological agent to biological tissue, said injector system, comprising:
a plurality of needles arranged in an array;
a plurality of reservoirs configured to receive a pharmacological composition therein, each of said reservoirs being in fluid communication with a respective one of said plurality of needles;
a pneumatic pressure source;
a pneumatic drive system in communication with said pressure source and said plurality of reservoirs, said pneumatic drive system being configured to induce a first discharge of said pharmacological composition out of said reservoirs and said plurality of needles in a plurality of delivery modes;
agent delivery volume control means for controlling volume of said pharmacological agent discharged from said plurality of needles; and
a control system, said control system including actuation control means for controlling said pneumatic drive system.

2. The injector system of claim 1, wherein said plurality of needles comprises 7 needles.

3. The injector system of claim 1, wherein each of said reservoirs is configured to receive at least 0.05 cc of said pharmacological composition.

4. The injector system of claim 1, wherein each of said reservoirs includes a reservoir piston disposed on a first end of each reservoir, each of said pistons having a first cross-sectional area.

5. The injector system of claim 1, wherein said pneumatic drive system includes a central drive piston spaced a first distance from each of said reservoir pistons, said drive piston having a second cross-sectional area.

6. The injector system of claim 5, wherein said drive piston second cross-sectional area and said first cross-sectional area of said reservoir pistons has a ratio of at least 0.5:1.

7. The injector system of claim 1, wherein said actuation control means is programmed to maintain actuation pressure provided by said pressure source in the range of 5-1000 psi.

8. The injector system of claim 1, wherein said delivery modes comprise continuous and pulsed delivery.

9. The injector system of claim 8, wherein said pulsed delivery has a frequency in the range of 0.1 Hz-5 kHz.

10. The injector system of claim 8, wherein said actuation control means is further programmed to control said delivery modes.

11. The injector system of claim 1, wherein said pharmacological composition comprises an extracellular matrix (ECM), said ECM composition including an ECM material selected from the group consisting of small intestine submucosa (SIS), urinary bladder submucosa (UBS), urinary basement membrane (UBM), liver basement membrane (LBM), stomach submucosa (SS), mesothelial tissue, subcutaneous extracellular matrix, large intestine extracellular matrix, placental extracellular matrix, ornamentum extracellular matrix, heart extracellular matrix and lung extracellular matrix.

12. The injector system of claim 11, wherein said ECM composition further includes at least one supplemental biologically active agent.

13. The injector system of claim 12, wherein said biologically active agent comprises a growth factor selected from the group consisting of a platelet derived growth factor (PDGF), epidermal growth factor (EGF), transforming growth factor-α (TGF-α), transforming growth factor-β (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor-α (TNA-α), and placental growth factor (PLGF).

14. The composition of claim 12, wherein said biologically active agent comprises a cell selected from the group consisting of a human embryonic stem cell, fetal cardiomyocyte, myofibroblast, mesenchymal stem cell, autotransplanted expanded cardiomyocytes, adipocyte, totipotent cell, pluripotent cell, blood stem cell, myoblast, adult stem cell, bone marrow cell, mesenchymal cell, embryonic stem cell, parenchymal cell, epithelial cell, endothelial cell, mesothelial cell, fibroblast, osteoblast, chondrocyte, exogenous cell, endogenous cell, hematopoietic stem cell, bone-marrow derived progenitor cell, myocardial cell, skeletal cell, fetal cell, undifferentiated cell, multi-potent progenitor cell, unipotent progenitor cell, monocyte, cardiac myoblast, skeletal myoblast, macrophage, capillary endothelial cell, xenogenic cell, allogenic cell and post-natal stem cell.

15. The composition of claim 12, wherein said biologically active agent comprises an active agent selected from the group consisting of a collagen (types I-V), proteoglycans, glycosaminoglycans (GAGs), glycoproteins, cytokines, cell-surface associated proteins, cell adhesion molecules (CAM), endothelial ligands, matrikines, cadherins, immuoglobins, fibril collagens, non-fibrallar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, finulin II, integrins, transmembrane molecules, thrombospondins, ostepontins, and angiotensin converting enzymes (ACE).
